(19) United States
(12) Patent Application Publication
(10) Pub. No.: US 2007/0254864 A1
(43) Pub. Date: Nov. 1, 2007

(54) CHROMIUM COMPLEXES

(75) Inventors: Richard W. Storrs, Berkeley, CA (US); Scott R. King, San Francisco, CA (US)

Correspondence Address: MORGAN, LEWIS & BOCKIUS LLP (SF), 2 PALO ALTO SQUARE, 3000 El Camino Real, Suite 700, PALO ALTO, CA, 94306, US

(73) Assignee: Mandrillus Pharma, LLC, San Francisco, CA (US)

(21) Appl. No.: 11/740,803

(22) Filed: Apr. 26, 2007

Related U.S. Application Data
(60) Provisional application No. 60/795,399, filed on Apr. 26, 2006.

(51) Int. Cl. A61K 31/555; C07F 11/00

(52) U.S. Cl. 514/184; 546/2

(57) ABSTRACT

The invention provides a complex which comprises at least one chromium ion and at least one pyridyl carboxylic acid moiety. The compounds of the invention are of use in controlling body mass and treating diseases caused by or characterized by deficiency of chromium metabolism, e.g., type 2 Diabetes, obesity, vascular disease and metabolic syndromes.

### CROSS-REFERENCE TO RELATED APPLICATIONS

This application is a non-provisional filing claiming priority to U.S. Provisional Patent Application No. 60/795,399, filed on Apr. 26, 2006, which is incorporated herein by reference in its entirety for all purposes.

### BACKGROUND OF THE INVENTION

Trivalent chromium (Cr³⁺) (“chromium”) is an essential trace element (Barrett et al., Polyhedron 4:1-14 (1985)) required for the maintenance of normal blood glucose (Mertz et al. J Am Coll Nutr 17: 544-547 (1998)) and fat metabolism (Abraham et al., Metabolism 41:768-771 (1992)). It is believed that chromium potentiates the action of insulin. The bioactive form was named glucose tolerance factor in the late 1950s (Schwartz et al., Arch Biochem Biophys 72:515-518 (1957)), a term that emphasizes its importance in glucose metabolism.

In general, it is accepted that a chromium intake of 30-40 μg/day is sufficient for achieving the daily requirements (Anderson et al., J Am Coll Nutr 17: 548-555 (1998)), and that healthy people usually reach these levels in their customary diet. However, because some foods, particularly those high in simple sugars, negatively affect the absorption and/or metabolism of chromium (Anderson et al., J Am Coll Nutr 17: 548-555 (1998)) and (Vincent et al., J Am Coll Nutr 18: 6-12 (1999)), in the absence of well-balanced diets, chromium deficiency may appear. Furthermore, because chromium metabolism is altered in diabetic subjects by increased loss, decreased absorption (Anderson et al., Diabetes 46:1786-1791 (1997)) and an inadequate dietary intake (Cefalu et al., J Nutr 132: 1107-1114 (2002)), little is known about the daily chromium requirements for those subjects.

Tyrosine kinase, an enzyme required for phosphorylation, is chromium dependent, and phosphotyrosine phosphatase, an enzyme that inactivates the insulin receptor, is inhibited by chromium (Anderson et al., J Am Coll Nutr 17: 548-555 (1998)). Thus, in addition to the increase in the number of insulin receptors (Anderson et al., J Am Coll Nutr 17: 548-555 (1998)), chromium improves the action of insulin by modulating tyrosine kinase activity on the insulin receptor (Cefalu et al, J Nutr 132: 1107-1114 (2002)), (Mertz e al., Nutr Rev 56: 174-177 (1998)) and (Davis et al., Biochemistry 36: 4382-4385 (1997)). Finally, it has been reported that Cr³⁺ also exerts a powerful cellular antioxidant action (Anderson et al., J Am Coll Nutr 20: 212-218 (2001)) and decreases the hepatic extraction of plasma insulin (Guan et al., J Nutr 130: 274-1279 (2000)). Deficiency of chromium may result in similar clinical manifestations to those observed in insulin resistance and type 2 diabetes, and supplementation with bioactive chromium could improve insulin sensitivity, leading to a more efficient peripheral glucose uptake.

As chromium seems to exert a positive effect on glucose and insulin levels of type 2 diabetic subjects, there is a need for new methods of delivering bioactive chromium, through new chromium complexes, to patients in need of such treatment. Moreover, as simple salts of chromium are poorly bioavailable, there is a need for chromium complexes with improved absorption or activity. This invention responds to this and other needs.

### SUMMARY OF THE INVENTION

The present invention provides novel chromium complexes formed between at least one ligand and a chromium ion. In an exemplary embodiment, the ligand forming a complex of the invention has the formula: 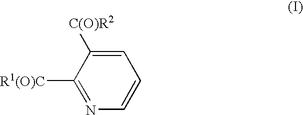
wherein R¹ and R² are members independently selected from OH and O⁻.

Also provided are pharmaceutical formulations including a complex of the invention. The invention also provides a method of using compounds of the invention to treat or ameliorate metabolic conditions caused by or related to abnormalities in chromium metabolism, e.g. abnormalities in glucose regulation. The compositions and methods of the invention are also of use to regulate body mass in a subject.

Other objects, aspects and advantages of the invention are apparent from the detailed description that follows.

### BRIEF DESCRIPTION OF THE DRAWINGS

FIG. 1 displays a liquid chromatography/mass spectrometry analysis of the complex made with 2:1 quinolinate:chromium in negative ion mode.

FIG. 2 displays a liquid chromatography/mass spectrometry analysis of the complex made with 3:1 quinolinate:chromium in positive ion mode.

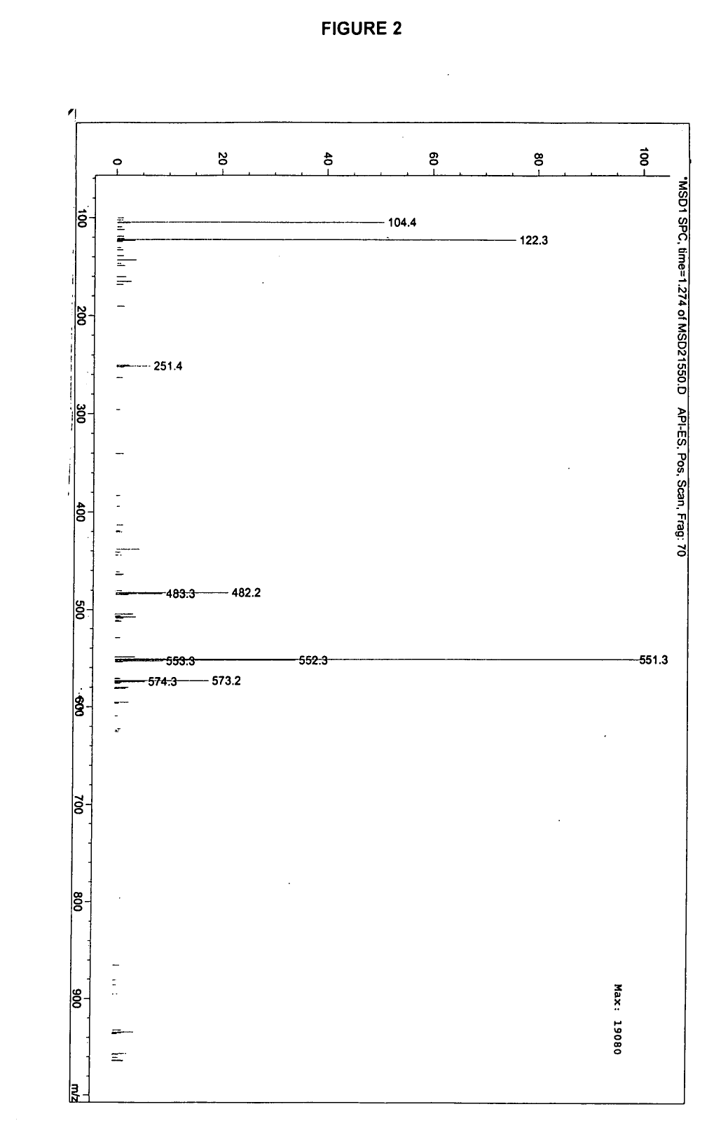

FIG. 3 displays the effect on body mass of administration of a compound of the invention at 1 mg/kg/day p.o. to db/db mice.

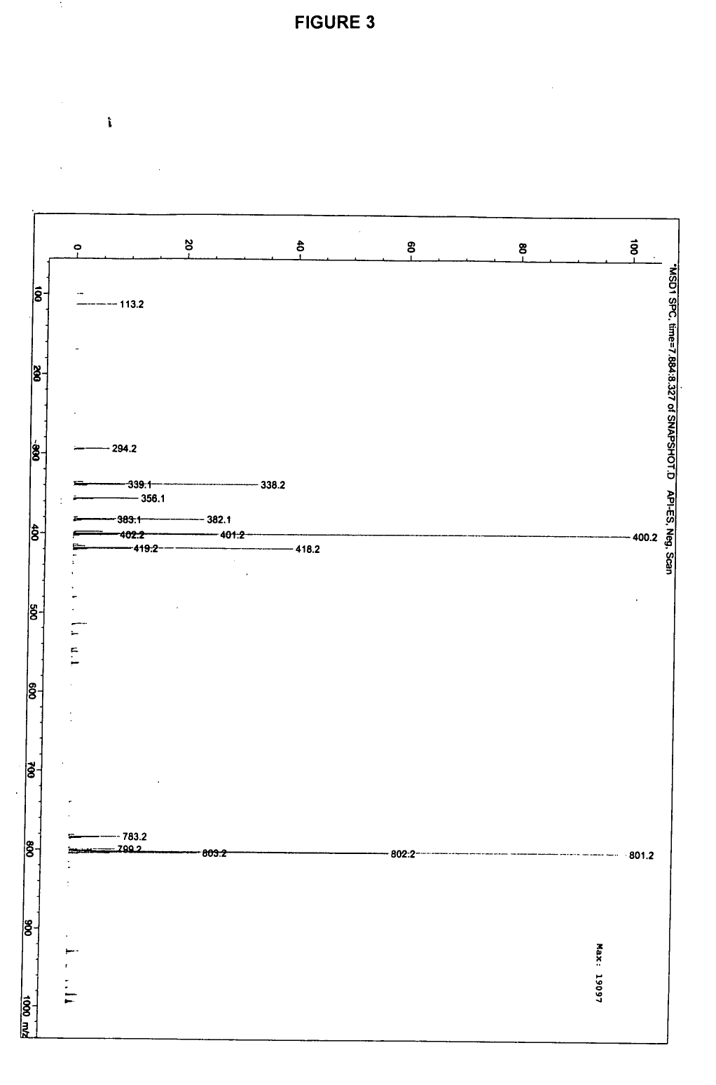

### DETAILED DESCRIPTION OF THE PREFERRED EMBODIMENTS

I. Definitions and Abbreviations

By “effective” amount of a drug, formulation, or permeant is meant a sufficient amount of a active agent to provide the desired local or systemic effect. A “pharmaceutically effective,” or “therapeutically effective” amount refers to the amount of drug needed to effect the desired therapeutic result.

The term “pharmaceutically acceptable salts” is meant to include salts of the compounds of the invention which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science 66: 1-19 (1977)). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compounds in the conventional manner. The parent form of the compound differs from the various salt forms in certain physical properties, such as solubility in polar solvents.

In addition to salt forms, the present invention provides compounds which are in a prodrug form. Prodrugs of the compounds or complexes described herein readily undergo chemical changes under physiological conditions to provide the compounds of the present invention. Additionally, prodrugs can be converted to the compounds of the present invention by chemical or biochemical methods in an ex vivo environment. Furthermore, the chromium chelates of the invention may inherently function as prodrugs, undergoing in vivo conversion to a species with properties improved over the chelate as administered, e.g., more active, more bioavailable, longer half life and the like.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), carbon-14 (¹⁴C) or chromium-51 (⁵¹Cr). All isotopic variations of the compounds of the present invention, whether radioactive or not, are intended to be encompassed within the scope of the present invention.

The term “pharmaceutically acceptable carrier” or “pharmaceutically acceptable vehicle” refers to any formulation or carrier medium that provides the appropriate delivery of an effective amount of a active agent as defined herein, does not interfere with the effectiveness of the biological activity of the active agent, and that is sufficiently non-toxic to the host or patient. Representative carriers include water, oils (e.g., vegetable and mineral). These bases can include suspending agents, thickeners, penetration enhancers, and the like. The carrier can also add enteric or controlled release characteristics to the compound of the invention. Their formulation is well known to those in the art of pharmaceuticals. Additional information concerning carriers can be found in Remington: The Science and Practice of Pharmacy, 21st Ed., Lippincott, Williams & Wilkins (2005) which is incorporated herein by reference.

The term “pharmaceutically acceptable additive” refers to preservatives, antioxidants, fragrances, emulsifiers, dyes and excipients known or used in the field of drug formulation and that do not unduly interfere with the effectiveness of the biological activity of the active agent, and that is sufficiently non-toxic to the host or patient. Additives for pharmaceutical formulations are well-known in the art, and may be added to the composition, as long as they are pharmaceutically acceptable and not deleterious to the subject. Further, they should not cause deterioration in the stability of the composition. For example, inert fillers, anti-irritants, tackifiers, excipients, fragrances, opacifiers, antioxidants, gelling agents, stabilizers, surfactants, coloring agents, preservatives, buffering agents, other permeation enhancers, and other conventional components of pharmaceutical formulations as are known in the art.

The term “excipients” is conventionally known to mean carriers, diluents and/or vehicles used in formulating drug compositions effective for the desired use.

II. Introduction

The present invention provides novel chromium complexes and methods for the preparation of these complexes. These compounds show activity in regulating metabolism that is enhanced relative to currently available dietary chromium supplements. Methods of using these chromium complexes to treat a condition characterized by an abnormality in glucose regulation or lipid metabolism, as well as pharmaceutical compositions comprising the chromium complexes are also described herein.

III. The Complexes

In a first aspect, the invention provides a complex between a chromium ion, e.g., Cr(III), and a heteroaryl carboxyl-containing ligand. The ligand and ion are present in any useful ratio. In a presently preferred embodiment, the ligand:ion ratio is 2:1 or 3:1. When more than one ligand complexes the chromium, the ligands can have the same structure or different structures. Thus, complexes with mixed ligand motifs are contemplated within the instant invention.

In another preferred embodiment, the ligand includes at least two moieties that include a carboxyl moiety.

In a preferred embodiment, a ligand forming a complex of the invention has the formula: 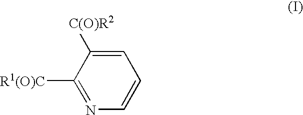
wherein R¹ and R² are members selected from OH and O⁻.

In another exemplary embodiment, the complex comprises one chromium ion and two ligand moieties. In yet another exemplary embodiment, the complex comprises one chromium ion and three ligand moieties.

A presently preferred ligand is pyridyl-2,3-dicarboxylic acid (quinolinic acid).

In a preferred embodiment, the complex is 2:1 or 3:1 quinolinate:chromium. Quinolinic acid is a mammalian metabolite of the essential amino acid tryptophan (Quinolinic Acid and the Kinurenines, Trevor W. Stone, ed., CRC Press 1989).

In compounds of the invention in which the hexadentate coordination sphere of the chromium ion is not completely satisfied by the pyridyl ligand according to Formula I, the vacancies in the coordination sphere are filled by one or more ligands other than that according to Formula (I). Exemplary ligands include OH, H₂O, NH₃, F, Cl, Br, I, carboxylic acids, alkyl substituted carboxylic acids, formate and acetate.

In another preferred embodiment, the chromium ion is present in the complex as prepared as Cr(III).

IIIa. The Pharmaceutical Compositions

In another aspect, the invention is a pharmaceutical formulation comprising: (a) a pharmaceutically acceptable carrier; and (b) a chromium complex of the invention.

The pharmaceutical formulations of the invention can take a variety of forms adapted to the chosen route of administration. Those skilled in the art will recognize various synthetic methodologies that may be employed to prepare non-toxic pharmaceutical formulations incorporating the compounds described herein. Those skilled in the art will recognize a wide variety of non-toxic pharmaceutically acceptable solvents that may be used to prepare solvates of the compounds of the invention, such as water, ethanol, propylene glycol, mineral oil, vegetable oil and dimethylsulfoxide (DMSO).

The compositions of the invention may be administered orally, parenterally, by inhalation or spray or rectally in dosage unit formulations containing conventional non-toxic pharmaceutically acceptable carriers, adjuvants and vehicles. It is further understood that the best method of administration may be a combination of methods. Oral administration in the form of a pill, capsule, elixir, syrup, lozenge, troche, or the like is particularly preferred. Enterically coated formulations are also provided by the present invention. The term parenteral as used herein includes subcutaneous injections, intradermal, intravascular (e.g., intravenous), intramuscular, intraperitoneal, spinal, intrathecal injection or like injection or infusion techniques.

The pharmaceutical formulations containing compounds of the invention are preferably in a form suitable for oral use, for example, as tablets, troches, lozenges, aqueous or oily suspensions, dispersible powders or granules, emulsion, hard or soft capsules, enteric coated formulations, syrups or elixirs.

Compositions intended for oral use may be prepared according to any method known in the art for the manufacture of pharmaceutical formulations, and such compositions may contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; and dispersing or wetting agents, which may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical formulations of the invention may also be in the form of oil-in-water emulsions and water-in-oil emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth; naturally-occurring phosphatides, for example soy bean, lecithin, and esters or partial esters derived from fatty acids and hexitol; anhydrides, for example sorbitan monooleate; and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The pharmaceutical formulations may be in the form of a sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The composition of the invention may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Exemplary materials are cocoa butter and polyethylene glycols and others.

The compositions of the invention can also be enterically coated. Depending upon the composition and/or thickness, the enteric coatings are resistant to stomach acid for selected periods of time before they begin to disintegrate and permit slow release of the drug in the lower stomach or upper part of the small intestines. Examples of some enteric coatings are previously employed are beeswax and glyceryl monostearate; beeswax, shellac and cellulose; and cetyl alcohol, mastic and shellac, as well as shellac and stearic acid (U.S. Pat. No. 2,809,918); polyvinyl acetate and ethyl cellulose (U.S. Pat. No. 3,835,221); and neutral copolymer of polymethacrylic acid esters (Eudragit L30D) (F. W. Goodhart et al., Pharm. Tech., pp. 64-71, April 1984); copolymers of methacrylic acid and methacrylic acid methylester (Eudragits), or a neutral copolymer of polymethacrylic acid esters containing metallic stearates (Mehta et al., U.S. Pat. Nos. 4,728,512 and 4,794,001).

Most enteric coating polymers begin to become soluble at pH 5.5 and above, with maximum solubility rates at pH greater than 6.5. A preferred enteric coating of use with the compounds of the invention will have these properties.

Alternatively, the compositions can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

For administration to non-human animals, the composition containing the therapeutic compound may be added to the animal's feed or drinking water. Also, it will be convenient to formulate animal feed and drinking water products so that the animal takes in an appropriate quantity of the compound in its diet. It will further be convenient to present the compound in a composition as a premix for addition to the feed or drinking water. The composition can also added as a food or drink supplement for humans.

Dosage levels of the order of from about 5 mg to about 250 mg per kilogram of body weight per day and more preferably from about 25 mg to about 150 mg per kilogram of body weight per day, are useful in the treatment of the above-indicated conditions. The amount of active ingredient that may be combined with the carrier materials to produce a single dosage form will vary depending upon the condition being treated and the particular mode of administration. Dosage unit forms will generally contain between from about 1 mg to about 500 mg of an active ingredient.

Frequency of dosage may also vary depending on the compound used and the particular disease treated. However, for treatment of most disorders, a dosage regimen of 4 times daily or less is preferred. It will be understood, however, that the specific dose level for any particular patient will depend upon a variety of factors including the activity of the specific compound employed, the age, body weight, general health, sex, diet, time of administration, route of administration and rate of excretion, drug combination and the severity of the particular disease undergoing therapy.

Preferred compounds of the invention will have desirable pharmacological properties that include, but are not limited to, oral bioavailability, low toxicity, low serum protein binding and desirable in vitro and in vivo half-lives.

Assays may be used to predict these desirable pharmacological properties. Assays used to predict bioavailability include transport across human intestinal cell monolayers, including Caco-2 cell monolayers. Toxicity to cultured hepatocyctes may be used to predict compound toxicity. Penetration of the blood brain barrier of a compound in humans may be predicted from the brain levels of laboratory animals that receive the compound intravenously.

Serum protein binding may be predicted from albumin binding assays. Such assays are described in a review by Oravcova, et al. (Journal of Chromatography B (1996) volume 677, pages 1-27).

Compound half-life is inversely proportional to the frequency of dosage of a compound. In vitro half-lives of compounds may be predicted from assays of microsomal half-life as described by Kuhnz and Gieschen (Drug Metabolism and Disposition, (1998) volume 26, pages 1120-1127).

The amount of the composition required for use in treatment will vary not only with the particular compound selected but also with the route of administration, the nature of the condition being treated and the age and condition of the patient and will ultimately be at the discretion of the attendant physician or clinician.

IV. The Methods

According to yet another aspect, the invention provides a method of treating, preventing, ameliorating or regulating a disease or condition. The method includes administering to a mammalian subject in need of treatment a therapeutically effective amount of a chromium complex of the invention.

In general, diseases and metabolic conditions that can be treated, ameliorated, regulated or prevented by administration of the compounds set forth herein include diseases that are caused by or characterized by a deficiency in chromium metabolism. Exemplary diseases according to this embodiment include diseases that are characterized by insulin resistance (or reduction in insulin sensitivity) in the subject including obesity, type 2 diabetes, type 1 diabetes, vascular disease, and metabolic syndromes.

In a preferred embodiment, the invention provides a method for controlling body mass in a mammal by administering to the mammalian subject an amount of a chromium complex sufficient to control the body mass. Diet-induced metabolic stresses may be ameliorated with compounds of the current invention.

As used herein a subject treated with a method for “controlling body mass” will have a body mass that is less than that of another subject of the same species who has not been treated with a chromium complex such as those set forth herein. The body mass is preferably measured after a selected period of treatment with the chromium complex. In a preferred embodiment, the treated subject has a body mass that is measurably lower than that of an untreated subject when body mass is measured after six months of treatment, preferably after four months of treatment, more preferably after three months of treatment and more preferably still after two months of treatment.

It is generally preferred that body mass is controlled by the method of the invention while food intake by the subject remains essentially unaltered relative to the subject's food intake prior to initiation of treatment by chromium complex administration. In a preferred embodiment, the food intake of the treated subject remains essentially unaltered for at least the first six months of treatment with a chromium complex such as those set forth herein.

As used herein, the term “essentially unaltered” refers to a food intake by the subject that is not more than 20%, preferably not more than 15%, more preferably not more than 10% and even more preferably not more than 5% different than the average daily food intake of the subject prior to beginning treatment with a chromium complex such as those set forth herein.

In another exemplary embodiment, body mass is controlled while blood sugar levels of a treated mammalian subject remain essentially identical to those of an untreated subject of the same species. As used herein, “essentially identical” refers to blood sugar levels that vary between the subjects by not more than about 10%, preferably not more than about 8%, more preferably not more than about 5%, and still more preferably not more than about 2% during at least the first six months of treatment with a chromium complex such as those set forth herein.

In another preferred embodiment, the method of the invention is of use in regulating body mass and/or glucose metabolism and/or insulin resistance in a patient who does not need to be treated with a chromium complex for another disease or deficiency.

In therapeutic use, the complexes of the invention are administered at the initial dosage of about 0.001 mg/kg to about 1000 mg/kg daily. A daily dose range of about 0.1 mg/kg to about 100 mg/kg is preferred, and a dose of from about 0.5 mg/kg to about 10 mg/kg being more preferred. The dosages, however, may be varied depending upon the requirements of the patient, the severity of the condition being treated, and the compound being employed. Determination of the proper dosage for a particular situation is within the skill of the practitioner.

Various aspects of the present invention will be further illustrated by the following non-limiting examples.

### EXAMPLES

### Example 1

Preparation of Chromium Quinolinate

Cr(H₂O)₆Cl₃ (50 mg), quinolinic acid (66 mg), and H₂O (5 mL) were comb heated by immersion in boiling water for one hour. The quinolinic acid slowly dissolved and the solution changed from a greenish to a purple-pink color. LC-MS demonstrated a product with apparent mass of 400 g/mol, consistent with a 2:1 quinolinate:chromium complex.

### Example 2

Preparation of Chromium Quinolinate

Cr(H₂O)₉(NO₃)₃ (75 mg), 66 mg quinolinic acid, and 5 mL H₂O were and heated by immersion in boiling water for one hour. The quinolinic acid slowly dissolved and the solution changed from a green to a purple-pink color. LC-MS demonstrated a product with apparent mass of 400 g/mol, consistent with a 2:1 quinolinate:chromium complex.

### Example 3

Preparation of Chromium Quinolinate

Cr(H₂O)₆Cl₃ (50 mg), quinolinic acid (132 mg), and 1M phosphate buffer pH 5.4 in H₂O (5 mL) were combined and heated by immersion in boiling water for one hour. The quinolinic acid slowly dissolved and the solution changed from a greenish to a purple-pink color. LC-MS demonstrated a product with apparent mass of 551 g/mol, consistent with 3:1 quinolinate:chromium complex.

### Example 4

Characterization of Chromium Quinolinate

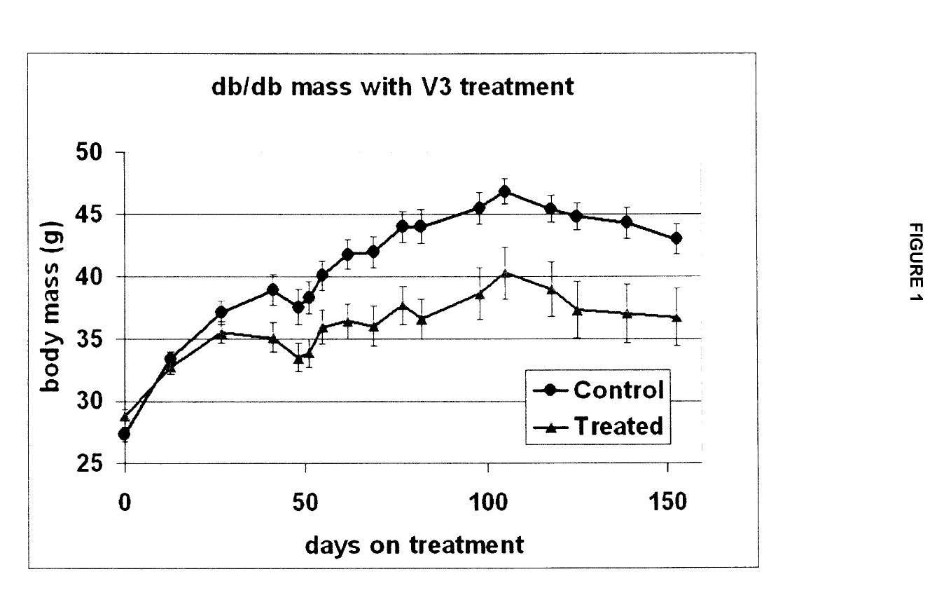

The material prepared in Example 1 was analyzed, following reverse-phase HPLC, using mass spectroscopy. The main HPLC peak intensities matched the molecular weight of CrQ₂ (where Q stand for quinolinic acid), CrQ₂-CO₂, CrQ₂+H₂O and CrQ₂+2H₂O. See FIG. 1. The reaction proceeded to high yield.

The mass spec peaks come in families of four which reflect the natural abundance of Cr isotopes and are diagnostic for same. This isotopic abundance is as follows: Cr(50): 4.35%, Cr(52): 83.79%, Cr(53): 9.50%, Cr(54): 2.36%. In the following analysis “families” of peaks are mentioned which are identified by the maximum intensity within the family corresponding to the Cr(52) isotope. “Q” is quinolinic acid, “P” is either 2- or 3-pyridinecarboxylic acid, two possible decarboxylation products of Q. A “⁻” indicates a mono-anion, created by the electrospray method. The 294 g/mol family corresponds to [CrQ₂-2CO₂]⁻, which is likely [CrP₂]⁻. The 338 g/mol family corresponds to [CrQ₂-CO₂]⁻, which is likely [CrQP]⁻. The 382 g/mol family corresponds to [Cr(Q-2H)₂]⁻. The 400 g/mol family corresponds to [CrQ₂.H₂O]⁻. The 418 g/mol family corresponds to [CrQ₂.2H₂O]⁻. The 801 g/mol family corresponds to {[Cr(Q-2H)]₂H.H₂O}⁻, a dimer of Cr(Q-2H). The minor 783 g/mol family is this dimer minus the water.

### Example 5

In Vivo Efficacy

In vivo efficacy of the chromium complexes in controlling body mass is demonstrated by feeding the compound of Example 1, diluted to 5 mM chromium with water and the pH adjusted to 5.8 with the addition of NaOH, ad libitum p.o. in the drinking water of five week old db/db mice (BKS.Cg-m+/+Lepr^{db}/J, Jackson Laboratories). The mice were otherwise fed normal chow. Over the course of 22 weeks, the mice consumed on average 1 mg/kg/day of the compound. Mice on treatment reached an average mass of 37 g, while control mice receiving water reached an average mass of 43 g.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A complex comprising a chromium ion complexed with at least one ligand having the formula: 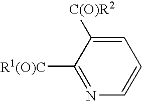
wherein R¹ and R² are members independently selected from OH and O⁻.

2. The complex according to claim 1 wherein said chromium ion is complexed by at least two of said ligand and R¹ and R² for each said ligand is independently selected.

3. The complex of claim 2, wherein said chromium ion is complexed by at least three of said ligand and R¹ and R² for each said ligand is independently selected.

4. The complex of claim 1, wherein at least one of said ligand is coordinated to chromium via the pyridine nitrogen.

5. The complex of claim 1, wherein said complex is chromium bis (pyridine-2,3-dicarboxylate).

6. The complex of claim 1, wherein said complex is chromium tris (pyridine-2,3-dicarboxylate).

7. A pharmaceutical formulation comprising a complex according to claim 1 and a pharmaceutically acceptable diluent.

8. A method of controlling body mass in a mammal, said method comprising:
administering to said mammal an amount of a complex according to claim 1 sufficient to control said body mass.

9. A method of treating a disease or condition characterized by insulin resistance, said method comprising:
administering to said mammal an amount of a complex according to claim 1 sufficient to treat said disease or condition.

10. The method according to claim 9, wherein said disease or condition is a member selected from type 1 diabetes and type 2 diabetes.

11. The method according to claim 9, wherein said disease or condition is obesity.
